(19) United States
(12) Patent Application Publication
(10) Pub. No.: US 2016/0310083 A1
(43) Pub. Date: Oct. 27, 2016

(54) RESPIRATORY MOTION COMPENSATION IN PHOTOACOUSTIC COMPUTED TOMOGRAPHY

(71) Applicant: WASHINGTON UNIVERSITY, St. Louis, MO (US)

(72) Inventors: Lihong WANG, St. Louis, MO (US); Jun XIA, St. Louis, MO (US); Konstantin MASLOV, St. Louis, MO (US); Wanyi CHEN, St. Louis, MO (US); Mark ANASTASIO, St. Louis, MO (US)

(73) Assignee: Washington University, St. Louis, MO (US)

(21) Appl. No.: 15/104,335

(22) PCT Filed: Dec. 22, 2014
(86) PCT No.: PCT/US2014/071967

§ 371 Date: Jun. 14, 2016

Related U.S. Application Data
(60) Provisional application No. 61/919,272, filed on Dec. 20, 2013.

(51) Int. Cl. A61B 5/00; A61B 5/113; A61B 5/0402

(52) U.S. Cl. CPC A61B 5/7207; A61B 5/0095; A61B 5/0402; A61B 5/113; A61B 5/0073; A61B 5/7285

(57) ABSTRACT

A respiratory-gated photoacoustic computed tomography system is disclosed that includes a respiratory motion sensor operatively coupled to a photoacoustic computed tomography system. The respiratory motion sensor monitors respiratory motion of a subject by measuring changes in the levels of a coupling fluid within which the subject is immersed.

### CROSS-REFERENCE TO RELATED APPLICATIONS

This application claims the benefit of U.S. Provisional Application No. 61/919,272, filed Dec. 20, 2013, the disclosure of which is hereby incorporated by reference herein in its entirety.

### GOVERNMENTAL RIGHTS IN THE INVENTION

This invention was made with government support under Grant No. R01 EB016963, awarded by the U.S. National Institutes of Health. The government has certain rights in the invention.

### FIELD OF THE INVENTION

The invention generally relates to systems and methods for improving image quality in photoacoustic computed tomography by reducing artifacts due to motion of the subject. In particular, the invention relates to systems and methods for reducing photoacoustic tomography image artifacts resulting from respiratory motion by the incorporation of a respiratory waveform obtained by monitoring respiratory displacement into an image reconstruction process.

### BACKGROUND OF THE INVENTION

Photoacoustic tomography is an emerging technique for preclinical whole-body imaging. PAT is based on the photoacoustic effect, which converts absorbed optical energy into pressure via thermoelastic expansion. The pressure waves generated by the absorption of optical energy are detected by ultrasonic transducers placed in one or more positions, and the complete dataset of pressure measurements are then processed to reconstruct a two-dimensional or three-dimensional image of the absorbed optical energy density in the tissue. The conversion of optical energy to acoustic waves enables PAT to generate high-resolution images in the optically diffusive regime.

In order to capture photoacoustic waves traveling along different directions, small-animal whole-body PAT systems typically have a large receiving aperture, achieved either by mechanically scanning a transducer array or by providing an array with a large number of elements. However, even in the latter case, due to the limited number of data acquisition channels, it is challenging to capture signals originating throughout the whole body of the subject with a single laser shot. Therefore, the majority of whole-body PAT systems have a frame rate of less than 1 Hz. Due to acoustic distortion from the lungs, whole-body PAT systems typically focus on regions below the chest, where respiration is the main cause of motion. Because a small-animal subject such as a mouse breathes at respiratory rates of up to 3 Hz, images acquired with a frame rate of less than 1 Hz are susceptible to respiratory motion artifacts. Respiratory gating, defined herein as the incorporation of respiratory motion into the capture and/or analysis of imaging data, may be essential in PAT as well as many other imaging applications, where accurate localization of organs is required. For instance, in high-intensity focused ultrasound (HIFU) tumor treatment, the respiration-induced organ displacement may be larger than the cross-sectional dimension of a focused treatment beam.

Because photoacoustic imaging requires a coupling medium, the subject is typically fully or partially immersed in a coupling medium. Typical coupling media used in photoacoustic imaging include water or aqueous solutions that are incompatible with the function of many types of electrical motion or respiratory sensors. Therefore, conventional electrical respiratory monitoring approaches, such as impedance pneumography, may be incompatible with PAT imaging. In addition, to ensure efficient transmission of both light and sound, opaque devices such as pressure sensors or strain gages cannot be mounted on the subject's body. Alternatively, one may use intubation and ventilation to precisely control the breathing cycle. However, that procedure requires special training, and repeated intubation for longitudinal monitoring may damage the subject's trachea or vocal cords.

Therefore, there is a need for accurate monitoring of an subject's respiratory waveform and reducing or compensating for respiratory motion in photoacoustic imaging to improve image quality.

### SUMMARY

In one aspect, a respiration-gated photoacoustic imaging device for obtaining photoacoustic computed tomography images of a region of interest of a subject immersed within a coupling medium is provided. The device may include a respiratory motion sensor to obtain a volume measurement of a combined volume consisting of a volume of the coupling medium and an immersed portion of the subject. The device may further include a light source to produce a light pulse directed into at least a portion of the region of interest of the subject. The light source may be operatively coupled to the respiratory motion sensor. The device may further include a transducer array to detect at least one photoacoustic signal produced by absorption of the light pulse within the region of interest. The device may further include a data processing device to: receive the at least one photoacoustic signal and an image volume measurement obtained at the same time as the at least one photoacoustic signal; reconstruct a photoacoustic image from the at least one photoacoustic signal; and record a photoacoustic image entry that may include the photoacoustic image and the image volume measurement. The device may obtain a plurality of photoacoustic image entries over at least one respiratory cycle of the subject. The data processing device may further combine at least two photoacoustic image entries with image volume measurements corresponding to essentially the same time within the respiratory cycle to create a photoacoustic computed tomography image of the region of interest of the subject. The image volume measurement may be triggered by the production of a light pulse by the light source. The data processing device may further analyze the image volume measurements of the plurality of photoacoustic image entries to identify the at least two photoacoustic image entries with volume measurements corresponding to essentially the same time within the respiratory cycle. The production of a light pulse by the light source may be triggered by the respiratory motion sensor when the volume measurement corresponding to a preselected time within the respiratory cycle is obtained. The respiratory motion sensor may be selected from the group consisting of: a float sensor; a hydrostatic sensor, a load cell sensor, a magnetic level sensor, a capacitance sensor, a time of flight sensor, and any combination thereof. The hydrostatic sensor may include a pressure sensor in fluid contact with the coupling medium via a tube in fluid contact with the coupling medium and with the pressure sensor at opposite ends. The tube may contain a fluid selected from the group consisting of: air, the coupling medium, and any combination thereof. The respiratory motion sensor may include an airflow sensor situated within an aerophore supplying air to the subject.

In another aspect, a method of obtaining a respiratory-gated photoacoustic computed tomography image of a region of interest of a subject immersed within a coupling medium is provided. The method may include monitoring a volume measurement of a combined volume consisting of a volume of the coupling medium and an immersed portion of the subject using a respiratory motion sensor. The method may also include detecting at least one photoacoustic signal produced by absorption of a light pulse within the region of interest using a transducer array, and reconstructing a photoacoustic image from the at least one photoacoustic signal. The method may also include determining a time within a respiratory cycle of the subject using an image volume measurement comprising the volume measurement obtained at the time at which the at least one photoacoustic signal is detected. The method may also include: recording a photoacoustic image entry comprising the photoacoustic image and the time within the respiratory cycle, and combining the photoacoustic image entry with at least one additional respiratory-gated photoacoustic image entry corresponding to essentially the same time in the respiratory cycle to form the respiratory-gated photoacoustic computed tomography image. The method may further include recording a plurality of additional photoacoustic image entries over at least one respiratory cycle of the subject. Each additional photoacoustic image entry may correspond to an additional time within the respiratory cycle of the subject. The method may further include determining a respiratory waveform of the subject that includes a set of reference volume measurements and a corresponding set of reference times within the respiratory cycle by analyzing the image volume measurement and a plurality of additional image volume measurements corresponding to the plurality of additional photoacoustic image entries. The method may further include selecting a portion of the plurality of additional photoacoustic image entries with essentially the same time in the respiratory cycle of the subject as the photoacoustic image entry as the at least one additional respiratory-gated photoacoustic image entry. The respiratory waveform may include about 20 reference volume measurements and corresponding reference times.

In an additional aspect, a method of obtaining a respiratory-gated photoacoustic computed tomography image of a region of interest of a subject immersed within a coupling medium is provided. The method may include monitoring a volume measurement of a combined volume consisting of a volume of the coupling medium and an immersed portion of the subject using a respiratory motion sensor and determining a time within a respiratory cycle of the subject based on the volume measurement. The method may further include triggering a light pulse when the time within the respiratory cycle matches a predetermined trigger time, and detecting at least one photoacoustic signal produced by absorption of the light pulse within the region of interest using a transducer array. The method may further include: reconstructing a photoacoustic image from the at least one photoacoustic signal; recording a photoacoustic image entry comprising the photoacoustic image and the time within the respiratory cycle; and combining the photoacoustic image entry with at least one additional respiratory-gated photoacoustic image entry corresponding to essentially the same predetermined trigger time to form the respiratory-gated photoacoustic computed tomography image. The time within the respiratory cycle may be determined by identifying a reference time within a respiratory waveform corresponding to the volume measurement. The respiratory waveform may include a set of reference volume measurements and a corresponding set of reference times within the respiratory cycle. The method may further include obtaining the respiratory waveform by analyzing a plurality of volume measurements obtained over at least one respiratory cycle of the subject. The respiratory waveform may include about 20 reference volume measurements and corresponding reference times.

While multiple embodiments are disclosed, still other embodiments of the present disclosure will become apparent to those skilled in the art from the following detailed description, which shows and describes illustrative embodiments of the disclosure. As will be realized, the invention is capable of modifications in various aspects, all without departing from the spirit and scope of the present disclosure. Accordingly, the drawings and detailed description are to be regarded as illustrative in nature and not restrictive.

### BRIEF DESCRIPTION OF THE FIGURES

The following figures illustrate various aspects of the disclosure.

FIG. 1 is a schematic diagram of the ring-shaped confocal photoacoustic computed tomography system with respiratory motion gating.

FIG. 2A is an in vivo mouse cross-sectional photoacoustic images acquired around the liver region without respiratory motion gating.

FIG. 2B is an in vivo mouse cross-sectional photoacoustic images acquired around the liver region with respiratory motion gating.

FIG. 2C is a graph summarizing temporal changes in the photoacoustic amplitude detected within the circled regions in FIG. 2A.

FIG. 2D is a graph summarizing temporal changes in the photoacoustic amplitude detected within the circled regions in FIG. 2B.

FIG. 3A is an in vivo mouse cross-sectional photoacoustic images acquired around the kidney region without respiratory motion gating.

FIG. 3B is an in vivo mouse cross-sectional photoacoustic images acquired around the kidney region with respiratory motion gating.

FIG. 3C is a graph summarizing temporal changes in the photoacoustic amplitude detected within the circled regions in FIG. 3A.

FIG. 3D is a graph summarizing temporal changes in the photoacoustic amplitude detected within the circled regions in FIG. 3B.

FIG. 4A is an in vivo mouse cross-sectional photoacoustic images acquired around the liver region with the hepatic vessels enlarged, obtained without respiratory motion gating.

FIG. 4B is an in vivo mouse cross-sectional photoacoustic images acquired around the liver region with the hepatic vessels enlarged, obtained with respiratory motion gating.

FIG. 4C is a graph summarizing temporal changes in the photoacoustic amplitude detected within the circled regions in FIG. 4A.

FIG. 4D is a graph summarizing temporal changes in the photoacoustic amplitude detected within the circled regions in FIG. 4B.

Corresponding reference characters and labels indicate corresponding elements among the views of the drawings. The headings used in the figures should not be interpreted to limit the scope of the claims.

### DETAILED DESCRIPTION

Provided herein are methods and systems for improving the quality of a photoacoustic tomography (PAT) image of a subject by incorporating respiratory motion measured for the subject while obtaining the PAT measurements of the subject. The systems and method may provide a simple and direct approach to recording a respiratory waveform based on a detected displacement of a coupling medium induced by the respiratory movements of a submerged portion of the subject, which may be a whole small animal. The recorded respiratory waveform may be used to reduce motion artifact in photoacoustic image reconstruction. The system and method may utilize a pressure sensor to detect respiratory motion within the coupling medium in an aspect.

In one aspect, the recorded respiratory waveform may be used to aid in the selection of a respiratory-gated set of photoacoustic images obtained during essentially the same time within the respiratory cycle. In various aspects, a retrospective respiratory gating method may be widely applicable to different whole-body PAT systems. The respiratory waveform may be recorded during photoacoustic data acquisition, where photoacoustic signals from different views may be captured at a constant speed. The entire dataset may then be sorted and clustered according to respiratory phases. The photoacoustic image may then be reconstructed using only data acquired from the same respiratory phase, greatly diminishing respiratory motion artifacts.

In another aspect, the respiratory motion of the subject may be continuously monitored and the recorded respiratory waveform may be used to trigger the acquisition of each member of the respiratory-gated set of photoacoustic images. In this other aspect, the detection of photoacoustic signals from different views may be triggered when the respiratory motion of the subject is at a preselected time within the respiratory cycle. This respiratory-gated set of photoacoustic images may be combined to form the respiratory-gated PAT image. A respiratory-gated PAT image may be obtained at additional pre-selected times within the respiratory cycle of the subject in a similar manner.

I. Photoacoustic Imaging System with Respiratory Gating

In an aspect, a system for respiratory gating for whole-body photoacoustic computed tomography of a subject is provided. The respiratory gating may be implemented in part by monitoring the respiratory motion of the subject using a novel respiratory motion sensor that makes use of the coupling fluid within which a subject of photoacoustic imaging may be submerged. In various aspects, changes in the level of the coupling fluid within a container holding both the coupling fluid and a submerged portion of the subject may be measured and used to deduce respiratory movements of the subject. Without being limited to any particular theory, it is assumed that changes in the combined volume of the coupling fluid and the submerged portion of the subject are due to changes in the volume of the subject's body associated with respiration.

In various aspects, the system for respiratory gating for whole-body photoacoustic computed tomography of a subject may include a photoacoustic tomography (PAT) system coupled to a respiratory motion sensor. In one aspect, a measurement of the respiratory motion may be obtained each time photoacoustic signals are obtained. In this one aspect, the measurement of the respiratory motion is essentially used as an index to identify the time within the respiratory cycle at which the PA image was obtained. In another aspect, the respiratory motion of the subject may be monitored and used to trigger the acquisition of a PA image at a preselected time within the respiratory cycle.

In various aspects, any PAT system that obtains PA images from a subject that is at least partially submerged in a coupling fluid may be included in the respiratory-gated PAT system without limitation. In various aspects, PAT systems configured to obtain PA images of whole small animals including, but not limited to, mice and rats may be operatively coupled with a respiratory motion sensor to effectuate the respiratory-gated PAT system as described herein. In one aspect, the PAT system may be a ring-shaped confocal PAT system.

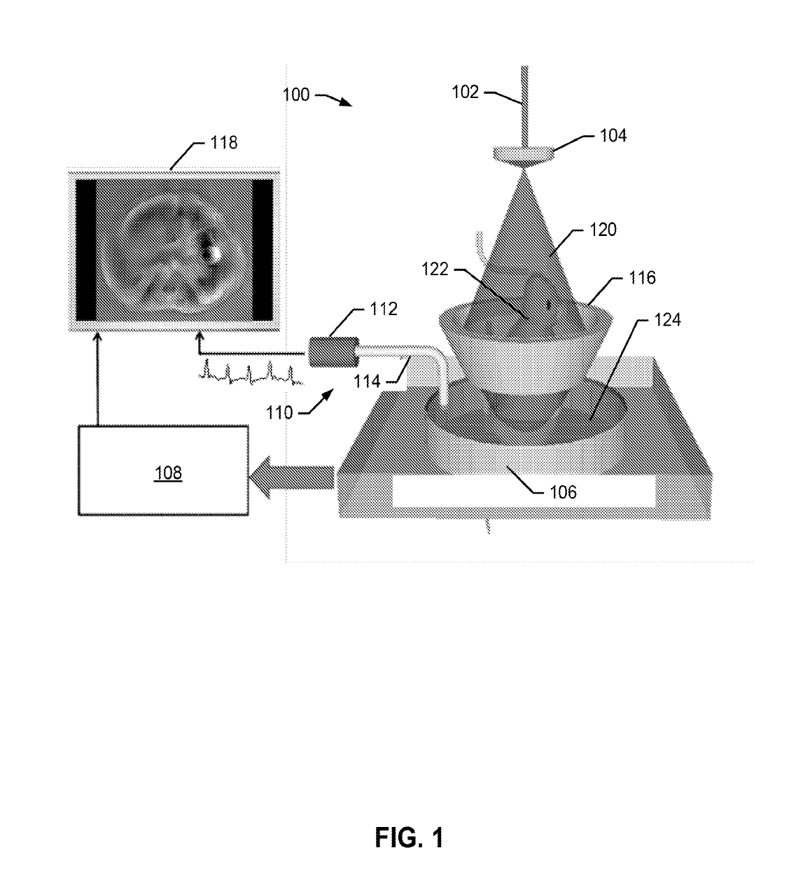

FIG. 1 is a schematic diagram of a respiratory-gated PAT system in one aspect that includes a ring-shaped confocal photoacoustic computed tomography (RC-PACT) system operatively coupled to a respiratory motion sensor. The respiratory-gated PAT system 100 may include a light source 102 including, but not limited to, a laser as well as associated optical components 104 including, but not limited to a conical lens to direct a light pulse 120 into the body of the subject 122. By way of non-limiting example, a 10-Hz pulse-repetition-rate Ti:sapphire laser may be used as the light source 102. The laser pulse may be first converted into a ring-shaped beam 120 by the conical lens and then redirected to the subject's body 122 by an optical condenser. Without being limited to any particular theory, each light pulse may illuminate structures within the region of interest of the subject 122, causing the region of interest to produce at least one photoacoustic signal.

Each of the at least one photoacoustic signals may include an ultrasound waveform induced by localized heating and expansion of structures caused by illumination of the structures by the light pulse 120. In this aspect, the photoacoustic signals may propagate through the coupling medium 124 and may be detected by a transducer array 106 in contact with the coupling medium 124. The transducer array 106 may be any known transducer array including, but not limited to, an array of unfocused and/or focused transducers in any known spatial arrangement including, but not limited to, a linear array, a planar array, a partial ring array, a full ring array, and any combination thereof.

In one aspect, the transducer array 106 may be a full ring transducer array as illustrated in FIG. 1. By way of non-limiting example, the photoacoustic signals may be detected by a 512-element full-ring transducer array with 5 MHz central frequency and more than 80% bandwidth.

In various aspects, the detected ultrasound signals are received by a data processing device 108, which reconstructs a PA image of the region of interest using various known image reconstruction methods. In one aspect, the data processing device may include a data acquisition system (DAQ) to receive the measurements from the transducer array 106. By way of non-limiting example, the transducer array data may be acquired by a 64-channel data acquisition system (DAQ) with 40 MHz sampling rate. Without being limited to any particular theory, the data transfer speed of the DAQ system may influence the sample rate of the system. By way of non-limiting example, in the system described herein above, the 40 Hz sampling rate of the DAQ system may result in the acquisition of transducer measurements associated with every other laser pulse, resulting in a full-ring transducer data acquisition time of about 1.6 seconds.

In various aspects, the respiratory-gated PAT system 100 may include a respiratory motion sensor 110 operatively coupled to the PAT system. In these various aspects, the respiratory motion sensor 110 may continuously monitor fluctuations in the coupling medium 124, which directly correlate to changes in the subject's corporeal volume. In an aspect, the coupling medium 124 may be water and the system 100 may take advantage of water coupling within the photoacoustic system. The inclusion of a respiratory motion sensor 110 to add respiratory-gating capability to a PAT system may be compatible with any whole-body PAT system without limitation, in particular those PAT systems that include immersion of the subject 122 into a coupling medium 124.

Any method for measuring the volume of a fluid in a container may be used to effectuate the respiratory motion sensor 110 without limitation. Non-limiting examples of suitable respiratory motion sensor devices include: a float sensor; a hydrostatic sensor, a load cell sensor, a magnetic level sensor, a capacitance sensor, a time of flight sensor, and any combination thereof.

In one aspect, the respiratory motion sensor 110 may be a float sensor. In various aspects, the float sensor may include a buoyant object formed from a material of lower density than that of the coupling medium 124. Without being limited to any particular theory, the buoyant object may translate vertically within a vessel containing the coupling fluid and the submerged portion of the subject as the height of the coupling fluid's surface changes during the respiratory movements of the subject. In an aspect, the float sensor may further include an additional material, including, but not limited to, a magnet to facilitate measuring the changes in the fluid level in a continuous manner.

In another aspect, the respiratory motion sensor 110 may be a hydrostatic sensor. Without being limited to any particular theory, the hydrostatic sensors measure changes in total pressure at a distance below the surface of the coupling fluid. As the surface of the coupling fluid rises and falls during the respiratory movements of the subject, the total pressure beneath the surface of the coupling fluid rises and falls proportionally. In various aspects, the hydrostatic sensor may include a displacer device, a bubbler device, and pressure sensor, and a differential-pressure sensor. In one aspect, the displacer device may include a column of a solid material with a density higher than the coupling medium. The solid column of the displacer device is suspended into the coupling fluid from an attachment above the surface of the coupling fluid that may include a force transducer. In this aspect, the displacer device may monitor changes in buoyance forces acting on the suspended column due to changes in the height of the coupling fluid due to respiratory motion.

In another aspect, the hydrostatic sensor may be a pressure sensor or a differential pressure sensor. In this other aspect, the pressure sensor may include a pressure transducer in contact with the coupling medium near the bottom of the vessel. In this aspect, the pressure transducer may measure fluctuations in total pressure resulting from increases or decreases in the depth of the coupling medium within the vessel induced by the subject's respiration. An additional pressure transducer may be situated above the surface of the coupling fluid and the pressure readings above and below the surface of the coupling fluid may be compared to provide a differential pressure measurement.

Referring again to FIG. 1, the respiratory motion sensor 110 may be a pressure sensor 112 operatively attached to a tube 114 with one end situated within the coupling medium in which the subject is submerged in an aspect. In this aspect, the tube may contain a fluid including, but not limited to, the coupling medium, air, and any combination thereof. In another aspect, the tube may contain air, and the pressure at the one end of the tube situated within the coupling medium may transfer to the pressure transducer situated at the opposite end via the air in the tube. As shown in FIG. 1, the input of the pressure sensor 112 may be connected to a plastic tube 114 at one end, and the opposite other end of the tube 114 may be immersed in the coupling medium, which compresses the air in the tube. Therefore, when the coupling medium level varies, the air pressure in the tube also changes. The output of the pressure sensor may be amplified and then digitized by a data processing system 108. In an aspect, the output may be digitized at a 100 Hz sampling rate. The output of the pressure sensor may further be used to determine respiratory phases and sort and cluster the photoacoustic dataset according to the respiratory phases to reduce motion artifacts in the reconstructed images as described herein.

In another aspect, the respiratory motion of the subject may be measured using an air-flow sensor. The respiratory waveform may be monitored using air-flow sensors installed near the inlet and outlet ports of an aerophore used to supply air to the subject. Respiration may induce flow changes superimposed on the bias air flow, which may be analyzed to provide inspiratory and expiratory phases versus time. Compared to intubation and ventilation, this approach may be non-invasive, allowing the subject to breathe on its own, and does not require special training to handle the subject.

Before image reconstruction, the photoacoustic data may be grouped according to the respiratory phase of the subject during data acquisition. In an aspect, the system may be used to develop an ultrasonic-image-based motion tracking technique with automated boundary identification.

In an aspect, the subject may include, but is not limited to, a small animal or a human. Non-limiting examples of suitable small animals include amphibians, fish, reptiles, birds, and mammals. Non-limiting examples of suitable mammals include mice, rats, rabbits, and humans.

II. Method for Photoacoustic Imaging with Respiratory Gating

Provided herein are methods for photoacoustic imaging with respiratory gating. The method may improve imaging quality under different respiratory rates and at multiple anatomical locations. Respiratory gating may also allow sorting and resampling of the data to a much higher frame rate, allowing visualization of the entire breathing cycle. In respiration-gated videos, the rhythmic movement of the liver, spleen and kidneys may be seen. Respiratory gating may also permit accurate tumor targeting during HIFU and radiation therapies.

This method involves simultaneous capturing of the subject's respiratory waveform and photoacoustic data acquisition. The recorded photoacoustic signals may be sorted and clustered according to the respiratory phase, and an image of the subject at each respiratory phase may be reconstructed subsequently from the corresponding cluster. In one aspect, the method may be used with a ring-shaped confocal photoacoustic computed tomography system. Respiratory gating may result in sharper vascular and anatomical images at different positions of the subject's body. In an aspect, the entire breathing cycle may be visualized at 20 frames per cycle.

In an aspect, the excitation laser may have a fixed triggering rate. In this aspect, retrospective respiratory gating may be used to group together data acquired at similar respiratory phases as described herein above. The fixed triggering rate may be about 10 Hz in one aspect.

In another aspect, using laser systems with flexible triggering options, prospective respiratory gating may be employed to trigger the laser to time data acquisition to a particular respiratory phase. The same data processing principle may be used for cardiac gating. Compared to image-based gating approaches, monitoring of the respiratory or cardiac waveform is immune to image noises and the data processing is computationally less intensive. Therefore, the method may be widely used to improve the image quality and broaden the applications of small-animal whole-body PAT.

Using a focused transducer array, the respiratory signal may be used for image averaging. The reconstructed cross-sectional images may be sorted into corresponding respiratory phases according to the waveform data and averaged for each respiratory phase. For 3D tomography using the unfocused transducer array, data over a complete respiratory cycle (about 1 s) may be acquired at each elevational position. The raw data may then be sorted based on the respiratory phases before 3D image reconstruction. With data collected from all respiratory phases, the subject's breathing may be visualized in three dimensions.

For cardiac imaging, the subject's cardiac cycle may be monitored using ECG. Electrodes may be installed on the subject's left and right forepaws. The ECG signal may be amplified by a differential amplifier and transferred to the computer. To minimize current leakage, deionized water may be used as the coupling medium. A cross section of the heart may be imaged continuously over several minutes, and then the data collected may be combined at the same respiratory phase to reconstruct images over a complete cardiac cycle.

In light of the fact that the skin boundary may be visualized in both photoacoustic and ultrasonic pulse-echo images, image-based motion tracking may be performed in which the respiratory waveform may be derived from the image sequence itself. An automated contour detection algorithm may trace the skin boundaries in PACT and USCT images. The total number of pixels within the contour line may then be calculated and plotted over time. The resulting curve may have a periodic oscillation similar to that in the waveform recorded by pressure or air-flow sensors, and may provide respiratory phase information.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

The system described herein previously and illustrated in FIG. 1 was used to obtain images of a mouse. In each experiment, 180 image frames were continuously acquired over a span of 4.8 minutes and then the data was sorted according to the respiratory waveform. For signals acquired at each laser shot, the temporal position was identified in the respiratory waveform and then assigned it a phase value, ranging from 0% to 100%, determined by its relative position between two adjacent respiratory peaks. The data were then sorted according to the respiratory phase and evenly clustered into 20 sets. The number of clusters was chosen to ensure that each cluster contained data from all 512 transducer elements, i.e., a full 27 imaging aperture was obtained. Due to 8:1 multiplexing, the full-ring array was divided into 8 segments, and each laser pulse generated data from one segment of the array. Within a cluster, different array segments may have produced data with different numbers of copies. Therefore, the data was averaged from each segment according to its number of copies, and then all segments were combined to form a single full-ring dataset, which was used to reconstruct a photoacoustic image for the given cluster. Merging images from all clusters produced a continuous video of the entire respiratory cycle. To mitigate image artifacts induced by acoustic reflectors, such as the spine and GI tract, the half-time image reconstruction principle was used. Because the main purpose of this study was to compensate for respiratory motion, rather than perform quantitative analysis, a non-iterative half-time reconstruction algorithm was employed that operated by directly back-projecting the first half of the raw data.

### Example 2

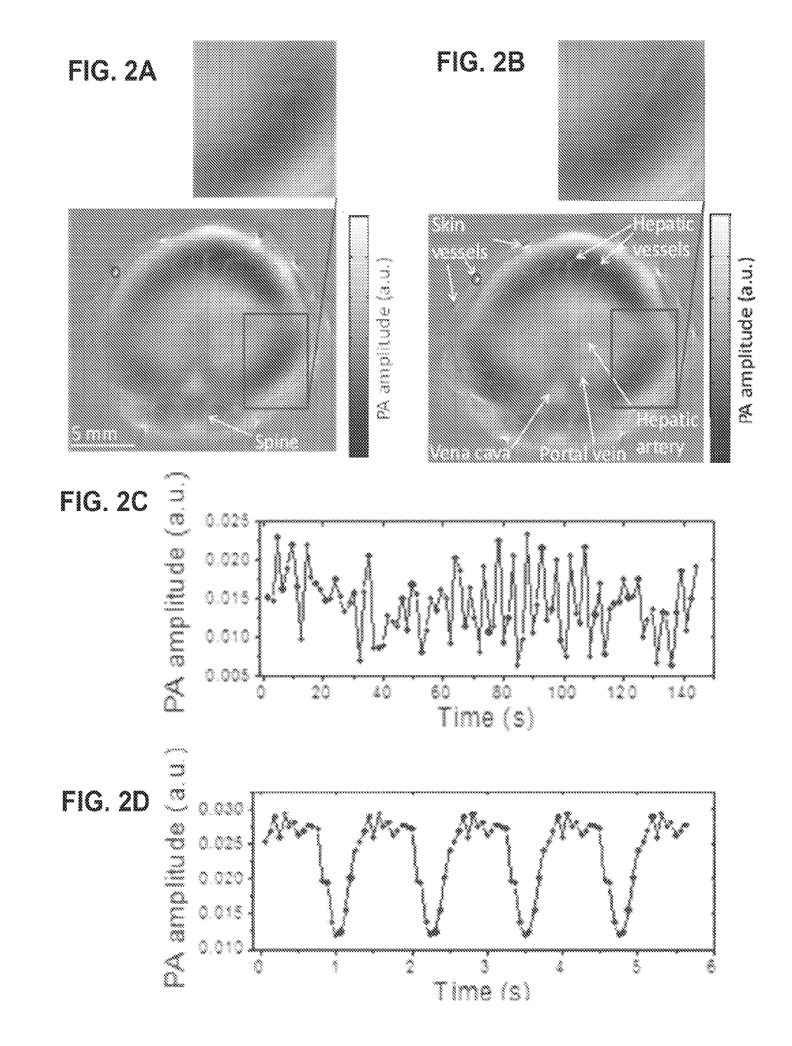

FIGS. 2A and 2B show in vivo cross sectional images acquired from the liver region of a 2-month-old nude mouse. FIG. 2A is an image reconstructed without respiratory motion gating. FIG. 2B is an image reconstructed with respiratory motion gating. The hepatic vessels in the box are enlarged to show the effect of respiratory motion correction. FIGS. 2C and 2D show temporal changes in photoacoustic amplitude within the circled regions in FIGS. 2A and 2B, respectively.

The mouse was anesthetized with isoflurane, which slowed its respiratory rate to 1.25 seconds per breath. Without motion compensation, each image frame was thus acquired over a period of 1.28 (i.e., 1.6/1.25) breathing cycles with 8 laser pulses. As expected, the ungated image (FIG. 2A) was appreciably more blurry than the gated image (FIG. 2B), especially for the hepatic vasculature. The skin boundary and cross sections of main blood vessels, such as vena cava, were also less clear in the ungated image due to the respiratory motion.

To better illustrate the respiratory effect, the temporal changes of photoacoustic amplitude were plotted from a small region marked with circles in FIGS. 2A and 2B. Both FIG. 2C and FIG. 2D contain data from 90 frames of images of the circled region. It may be seen that respiratory gating not only allowed visualization of the breathing cycle coherently but also improved the temporal resolution. In FIG. 2D, the amplitude drop with body expansion, which moved the skin vessel out of the circled region, may be seen. In contrast, FIG. 2C shows only randomized amplitude fluctuation. The rhythmic respiratory expansion and contraction of the subject's body was seen. It should be noted that the resampled 15.4-Hz (i.e., 1/0.065 s) frame rate was faster than the 10-Hz laser pulse repetition rate. This improvement was due to both the non-integer ratio of the laser's pulse repetition rate (10 Hz) to the respiratory rate (˜0.8 Hz) and the fluctuation of the respiratory cycle (±4% over the imaging period).

### Example 3

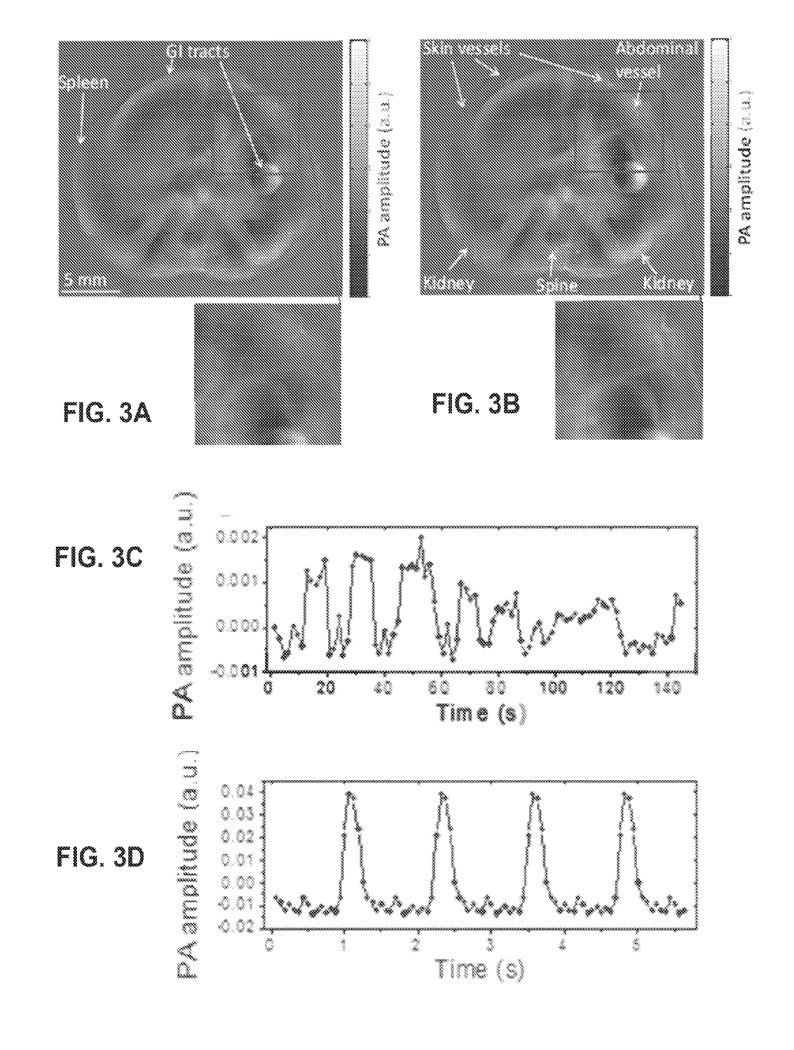

The kidney region was also imaged, where more organs may be visualized. FIGS. 3A and 3B are in vivo small-animal cross-sectional photoacoustic images acquired around the kidney region. FIG. 3A is an image reconstructed without respiratory motion gating. FIG. 3B is an image reconstructed with respiratory motion gating. The abdominal vessels are enlarged to show the effect of respiratory motion correction. FIGS. 3C and 3D show temporal changes in photoacoustic amplitude within the circled regions in FIGS. 3A and 3B, respectively.

While the kidneys were farther away from the lungs than the liver, the effect of respiratory motion was still evident. The kidneys, spleen, spine, and vascular network in the uncorrected FIG. 3A were more blurred than the counterparts in the motion-compensated FIG. 3B. The skin and abdominal vessels were also difficult to identify in FIG. 3A. In FIGS. 3C and 3D, the temporal photoacoustic signal changes within a circle placed in between the skin and spleen. In FIG. 3D, the signal increased due to body expansion, which moved the spleen to the circled region. The rhythmic respiratory motion of the subject's body, as well as the movements of its organs, were clearly observed.

### Example 4

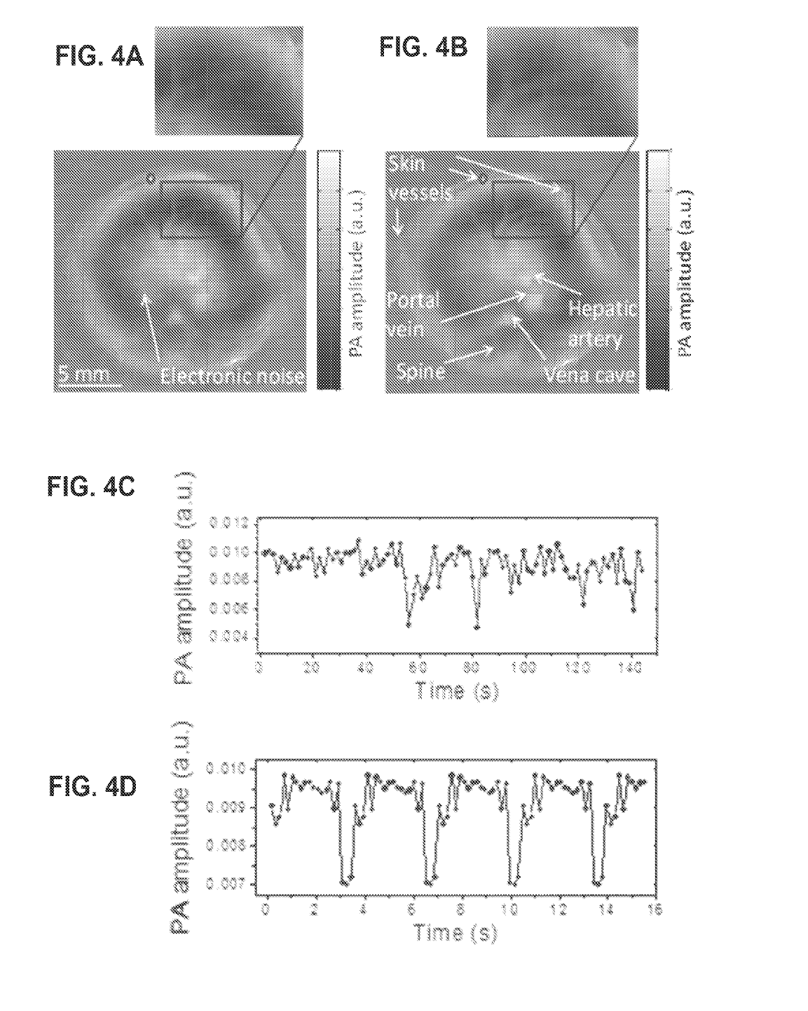

The rate of the subject's respiration was slowed by increasing the isoflurane concentration in the inhalation gas. In another study, a mouse with a respiratory rate of approximately 0.31 Hz was imaged, which was slower than the imaging frame rate (0.625 Hz). FIGS. 4A and 4B are in vivo small-animal cross-sectional photoacoustic images acquired around the liver region. FIGS. 4A and 4B compare the ungated and gated images, respectively. While the blurs caused by the respiratory motion were not as obvious as in the previous Examples, the hepatic vascular structures were still visualized more clearly in the motion-compensated image (FIG. 4B). In addition, the signal-to-noise ratio was also improved, as may be seen from the disappearance of the system's electronic-noise artifact in FIG. 4B. Because each gated image was an average of approximately 9 (180/20) projections, the signal to noise ratio was improved by 3 times. Therefore, even when the respiratory rate was slower than the imaging frame rate, respiratory gating was still beneficial. FIGS. 4C and 4D show changes in photoacoustic amplitude within the circles in FIGS. 4A and 4B, respectively. As expected, FIG. 4D showed periodic drop in photoacoustic amplitude due to body expansion, which moved the skin vessel out of the circled region. Compared to FIG. 2D, FIG. 4D had a longer resting period between breaths. This phenomenon is commonly observed in respiratory depression caused by high isoflurane concentrations.

The foregoing merely illustrates the principles of the invention. Various modifications and alterations to the described embodiments will be apparent to those skilled in the art in view of the teachings herein. It will thus be appreciated that those skilled in the art will be able to devise numerous systems, arrangements and methods which, although not explicitly shown or described herein, embody the principles of the invention and are thus within the spirit and scope of the present invention. From the above description and drawings, it will be understood by those of ordinary skill in the art that the particular embodiments shown and described are for purposes of illustrations only and are not intended to limit the scope of the present invention. References to details of particular embodiments are not intended to limit the scope of the invention.

What is claimed is:

1. A respiration-gated photoacoustic imaging device for obtaining photoacoustic computed tomography images of a region of interest of a subject immersed within a coupling medium, the device comprising:
a respiratory motion sensor to obtain a volume measurement of a combined volume consisting of a volume of the coupling medium and an immersed portion of the subject;
a light source to produce a light pulse directed into at least a portion of the region of interest of the subject, wherein the light source is operatively coupled to the respiratory motion sensor;
a transducer array to detect at least one photoacoustic signal produced by absorption of the light pulse within the region of interest; and
a data processing device to:
receive the at least one photoacoustic signal and an image volume measurement obtained at the same time as the at least one photoacoustic signal;
reconstruct a photoacoustic image from the at least one photoacoustic signal; and
record a photoacoustic image entry comprising the photoacoustic image and the image volume measurement.

2. The device of claim 1, wherein the device obtains a plurality of photoacoustic image entries over at least one respiratory cycle of the subject.

3. The device of claim 2, wherein the data processing device further combines at least two photoacoustic image entries with image volume measurements corresponding to essentially the same time within the respiratory cycle to create a photoacoustic computed tomography image of the region of interest of the subject.

4. The device of claim 3, wherein the image volume measurement is triggered by the production of a light pulse by the light source.

5. The device of claim 4, wherein the data processing device further analyzes the image volume measurements of the plurality of photoacoustic image entries to identify the at least two photoacoustic image entries with volume measurements corresponding to essentially the same time within the respiratory cycle.

6. The device of claim 3, wherein the production of a light pulse by the light source is triggered by the respiratory motion sensor when the volume measurement corresponding to a preselected time within the respiratory cycle is obtained.

7. The device of claim 1, wherein the respiratory motion sensor is selected from the group consisting of: a float sensor; a hydrostatic sensor, a load cell sensor, a magnetic level sensor, a capacitance sensor, a time of flight sensor, and any combination thereof.

8. The device of claim 7, wherein hydrostatic sensor comprises a pressure sensor in fluid contact with the coupling medium via a tube in fluid contact with the coupling medium and with the pressure sensor at opposite ends.

9. The device of claim 8, wherein the tube contains a fluid selected from the group consisting of: air, the coupling medium, and any combination thereof.

10. The device of claim 1, wherein the respiratory motion sensor comprises an airflow sensor situated within an aerophore supplying air to the subject.

11. A method of obtaining a respiratory-gated photoacoustic computed tomography image of a region of interest of a subject immersed within a coupling medium, the method comprising:
monitoring a volume measurement of a combined volume consisting of a volume of the coupling medium and an immersed portion of the subject using a respiratory motion sensor;
detecting at least one photoacoustic signal produced by absorption of a light pulse within the region of interest using a transducer array;
reconstructing a photoacoustic image from the at least one photoacoustic signal;
determining a time within a respiratory cycle of the subject using an image volume measurement comprising the volume measurement obtained at the time at which the at least one photoacoustic signal is detected;
recording a photoacoustic image entry comprising the photoacoustic image and the time within the respiratory cycle;
combining the photoacoustic image entry with at least one additional respiratory-gated photoacoustic image entry corresponding to essentially the same time in the respiratory cycle to form the respiratory-gated photoacoustic computed tomography image.

12. The method of claim 11, further comprising recording a plurality of additional photoacoustic image entries over at least one respiratory cycle of the subject, each additional photoacoustic image entry corresponding to an additional time within the respiratory cycle of the subject.

13. The method of claim 12, further comprising determining a respiratory waveform of the subject comprising a set of reference volume measurements and a corresponding set of reference times within the respiratory cycle by analyzing the image volume measurement and a plurality of additional image volume measurements corresponding to the plurality of additional photoacoustic image entries.

14. The method of claim 13, further comprising selecting a portion of the plurality of additional photoacoustic image entries with essentially the same time in the respiratory cycle of the subject as the photoacoustic image entry as the at least one additional respiratory-gated photoacoustic image entry.

15. The method of claim 14, wherein the respiratory waveform comprises about 20 reference volume measurements and corresponding reference times.

16. A method of obtaining a respiratory-gated photoacoustic computed tomography image of a region of interest of a subject immersed within a coupling medium, the method comprising:
monitoring a volume measurement of a combined volume consisting of a volume of the coupling medium and an immersed portion of the subject using a respiratory motion sensor;
determining a time within a respiratory cycle of the subject based on the volume measurement;
triggering a light pulse when the time within the respiratory cycle matches a predetermined trigger time;
detecting at least one photoacoustic signal produced by absorption of the light pulse within the region of interest using a transducer array;
reconstructing a photoacoustic image from the at least one photoacoustic signal;
recording a photoacoustic image entry comprising the photoacoustic image and the time within the respiratory cycle;
combining the photoacoustic image entry with at least one additional respiratory-gated photoacoustic image entry corresponding to essentially the same predetermined trigger time to form the respiratory-gated photoacoustic computed tomography image.

17. The method of claim 16, wherein the time within the respiratory cycle is determined by identifying a reference time within a respiratory waveform corresponding to the volume measurement, the respiratory waveform comprising a set of reference volume measurements and a corresponding set of reference times within the respiratory cycle.

18. The method of claim 17, further comprising obtaining the respiratory waveform by analyzing a plurality of volume measurements obtained over at least one respiratory cycle of the subject.

19. The method of claim 16, wherein the respiratory waveform comprises about 20 reference volume measurements and corresponding reference times.
